(19) United States
(12) Patent Application Publication
(10) Pub. No.: US 2011/0256193 A1
(43) Pub. Date: Oct. 20, 2011

(54) Use Of Copolymers Based On Polyethers And Vinyl Monomers As Stabilizing Agents For Emulsions

(75) Inventors: Kathrin Meyer-Bohm, Feucht (DE); Rainer Dobrawa, Stuttgart (DE); Karl Kolter, Limburgerhof (DE)

(73) Assignee: BASF SE

(21) Appl. No.: 13/141,481

(22) PCT Filed: Dec. 10, 2009
(86) PCT No.: PCT/EP2009/066784

§ 371 Date: Jun. 22, 2011

(30) Foreign Application Priority Data
Dec. 23, 2008 (EP) 08172744.8

(51) Int. Cl. A61K 8/06; A61K 9/107; A61K 47/32

(52) U.S. Cl. 424/401; 514/772.5; 424/400

(57) ABSTRACT

Use of water-soluble or water-dispersible copolymers which are obtained by polymerization of vinyl acetate and N-vinyllactams in the presence of a polyether, as stabilizing agents for emulsions.

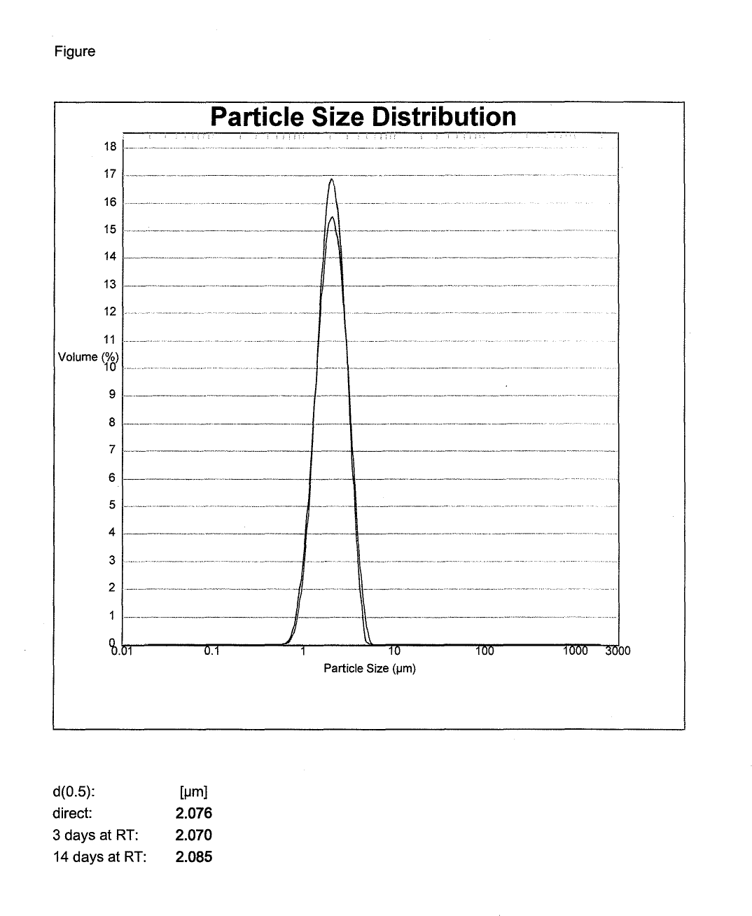

The present invention relates to the use of copolymers which are obtained by polymerization of vinyl acetate and N-vinyllactams in the presence of a polyether, as stabilizing agents for emulsions. The invention further relates to the corresponding emulsions and to pharmaceutical and cosmetic preparations based on these emulsions.

Emulsions are disperse systems of two immiscible liquids. Here, a distinction is made between the internal or dispersed phase, which is distributed in discrete droplets, and the external phase, the dispersant (coherent phase). A distinction is made between oil-in-water (O/W) emulsions and water-in-oil (W/O) emulsions, depending on the phase position of the system in question.

Emulsions are used particularly in the field of pharmaceutical and cosmetic preparation.

One problem of emulsions, however, is their tendency towards instability, particularly upon storage over a prolonged period. A growing-together of the droplets of the internal phase, which is referred to as coalescence, can result in a coarsening of the emulsion ranging to the complete collapse of the emulsion with complete phase separation. It was therefore the object to provide novel and improved stabilizing agents.

Emulsifiers often used hitherto are, for example, anionic surfactants such as sodium dodecyl sulfate, or sodium dioctyl sulfosuccinate, amphoteric surfactants such as, for example, lecithin, nonionic surfactants such as, for example, cetyl alcohol, cetylstearyl alcohol, stearyl alcohol, sorbitan fatty acid esters, polyoxyethylene sorbitan fatty acid esters, polyoxyethylene fatty acid glycerides, polyoxyethylene fatty acid esters or fatty acid ethers, glycerol fatty acid esters or macromolecular surfactants such as the poloxamers or macrogols.

WO 2007/051743 discloses the use of copolymers which are obtained by polymerization of N-vinyllactam, vinyl acetate and polyethers, as solubility promoters for active ingredients which are sparingly soluble in water.

The object was achieved according to the invention through the use of water-soluble or water-dispersible copolymers which are obtained by radically initiated polymerization of a mixture of
i) 30 to 80% by weight of N-vinyllactam,
ii) 10 to 50% by weight of vinyl acetate and
iii) 10 to 50% by weight of a polyether,
   with the proviso that the sum of i), ii) and iii) is equal to 100% by weight, as binders for producing solid active-ingredient-containing dosage forms.

Preferred polymers are obtained from:
i) 30 to 70% by weight of N-vinyllactam
ii) 15 to 35% by weight of vinyl acetate, and
iii) 10 to 35% by weight of a polyether.

Particularly preferred polymers are obtained from:
i) 40 to 60% by weight of N-vinyllactam
ii) 15 to 35% by weight of vinyl acetate
iii) 10 to 30% by weight of a polyether.

The proviso that the sum of components i), ii) and iii) is equal to 100% by weight also applies for the preferred and particularly preferred compositions.

Suitable as N-vinyllactam are N-vinylcaprolactam or N-vinylpyrrolidone or mixtures thereof. Preference is given to using N-vinylcaprolactam.

Suitable polyethers are preferably polyalkylene glycols. The polyalkylene glycols can have molecular weights of from 1000 to 100 000 D [daltons], preferably 1500 to 35 000 D, particularly preferably 1500 to 10 000 D. The molecular weights are determined starting from the OH number measured in accordance with DIN 53240.

Suitable particularly preferred polyalkylene glycols are polyethylene glycols.

Polypropylene glycols, polytetrahydrofurans or polybutylene glycols which are obtained from 2-ethyloxirane or 2,3-dimethyloxirane are also suitable.

Suitable polyethers are also random or block-like copolymers of polyalkylene glycols obtained from ethylene oxide, propylene oxide and butylene oxides, such as, for example, polyethylene glycol-polypropylene glycol block copolymers. The block copolymers can be of the AB or ABA type.

Preferred polyalkylene glycols also include those which are alkylated at one or both OH end groups. Suitable alkyl radicals are branched or unbranched C₁- to C₂₂-alkyl radicals, preferably C₁-C₁₈-alkyl radicals, for example methyl, ethyl, n-butyl, isobutyl, pentyl, hexyl, octyl, nonyl, decyl, dodecyl, tridecyl or octadecyl radicals.

General methods for preparing the copolymers according to the invention are known per se. The preparation takes place by radically initiated polymerization, preferably solution polymerization, in non-aqueous, organic solvents or in mixed non-aqueous/aqueous solvents. The preparation of the copolymers and their conversion to the powder form is described in detail in WO 2007/051743, to the disclosure of which with regard to the preparation of the copolymers reference is hereby expressly made.

The polymers have K values in accordance with Fikentscher in the range from 10 to 60, preferably 15 to 40, measured in a 1% strength by weight ethanolic solution.

According to the invention, the polymers are suitable for use as stabilizing agents for emulsions, in particular for oil-in-water emulsions.

The fraction of stabilizing agents in the emulsion can be 1 to 50, preferably 2 to 40, particularly preferably 5 to 25% by weight.

Emulsions can be prepared by the following part steps:
preparation of the lipophilic phase and of the hydrophilic phase
dispersion of the immiscible phases
homogenization.

In the case of the phase-inversion process, the phase which is to be the disperse phase in the finished emulsion is introduced as initial charge. The emulsifiers can then either be distributed to both phases, or else incorporated all together into the phase introduced as initial charge.

According to a further process, a base emulsion can firstly be prepared, into which the remaining hydrophilic phase can be incorporated.

A further process for preparing emulsions is carried out in such a way that the emulsifier is incorporated into the coherent phase and this is gradually incorporated the disperse phase.

Of suitability for the homogenization are the devices customary for this, such as paddle stirrer, rod stirrer or Ultra-Turrax.

The mixing processes can be carried out at room temperature. However, it may also be advisable to heat the mixtures and then to homogenize them. The person skilled in the art can establish which method is most suitable in the individual case by means of a few simple experiments.

Such emulsions are suitable in particular for pharmaceutical or cosmetic compositions. However, it is also possible to stabilize emulsions for technical applications.

Emulsions according to the invention are suitable for pharmaceutical or cosmetic preparations of active ingredients which are original oils, or preparations of oil-soluble active ingredients. It is of course also possible to incorporate other active ingredients either in dissolved or dispersed form.

Original oils are, for example:

jojoba oil, coconut oil, almond oil, olive oil, palm oil, castor oil, soybean oil or wheat germ oil or for essential oils such as dwarf pine needle oil, lavender oil, rosemary oil, spruce needle oil, pine needle oil, eucalyptus oil, peppermint oil, sage oil, bergamot oil, turpentine oil, melissa oil, juniper oil, lemon oil, anise oil, cardamom oil; peppermint oil, camphor oil etc. or for mixtures of these oils.

Suitable active ingredients are, for example, the corticoids such as hydrocortisone.

If desired, further pharmaceutically customary auxiliaries can be used in amounts of from 0 to 15% by weight.

The pharmaceutical preparations can be used customary pharmaceutical auxiliaries such as antioxidants, preservatives, thickeners, chelating agents, odorous substances, pH regulators, for example acidifying agents, buffer substances, sweeteners, aromas, flavor enhancers and dyes. The following substances are particularly suitable here: citric acid, tartaric acid, ascorbic acid, sodium dihydrogenphosphate, cyclamate, saccharin-Na, aspartam, menthol, peppermint aroma, fruit aromas, vanilla aroma, glutamate, riboflavin, betacarotene, water-soluble dyes, finely divided colored lakes. Suitable thickeners are, for example, methylcellulose, hydroxypropylmethylcellulose, hydroxypropylcellulose, hydroxyethylcellulose, carrageenans, pectins, xanthans or alginates be used.

Furthermore, additional interface-active compounds can also be added, for example sodium lauryl sulfate, dioctyl sulfosuccinate, alkoxylated sorbitan esters such as polysorbate 80, polyalkoxylated derivatives of castor oil or hydrogenated castor oil, for example Cremophor® RH 40, alkoxylated fatty acids, alkoxylated hydroxy fatty acids, alkoxylated fatty alcohols, alkali metal salts of fatty acids and lecithins.

Furthermore, pigments such as iron oxides, titanium dioxide, colloidal or precipitated silica, calcium carbonates or calcium phosphates can also be added.

The emulsions according to the invention of the oil-in-water type can be used for pharmaceutical preparations in the form of creams, lotions or milk-like liquid preparations.

Furthermore, according to the invention, the specified copolymers are also suitable as stabilizing agents for cosmetic preparations. As a rule, the cosmetic preparation is used for topical application to the skin.

In general, cosmetic and/or dermatological preparations are understood as meaning mixtures or formulations which are used for topical application on skin or hair and which are suitable (i) for preventing damage to human skin and/or human hair, (ii) for treating existing damage to human skin and/or human hair, (iii) for caring for human skin and/or human hair and/or (iv) for improving the skin feel (sensory properties). Explicitly comprised are compositions for decorative cosmetics. The cosmetic and/or dermatological preparations described in the process according to the invention are preparations whose main indication is primarily (e.g. in the case of sunscreen preparations) or else inter alia (e.g. in the case of daycare products, in antiaging products, in self-tanning preparations) the protection of skin against damage by sunlight, specifically by UV-B radiation (280 to 320 nm) and UV-A radiation (>320 nm). Besides the UV filter substances in a cosmetically compatible medium, the cosmetic and/or dermatological preparations comprise suitable auxiliaries and additives, which are selected with regard to the specific field of application. Auxiliaries and additives of this type are known to the person skilled in the art and can be found e.g. in cosmetics handbooks, for example Schrader, Grundlagen und Rezepturen der Kosmetika [Fundamentals and formulations of cosmetics], Hüthig Verlag, Heidelberg, 1989, ISBN 3-7785-1491-1, or Umbach, Kosmetik: Entwicklung, Herstellung und Anwendung kosmetischer Mittel [Cosmetics: development, preparation and use of cosmetic compositions], 2nd expanded edition, 1995, Georg Thieme Verlag, ISBN 3 13 712602 9.

The term UV-A filters is understood as meaning in particular oil-soluble or water-soluble substances, but also sparingly soluble or pigment-like substances, the absorption maximum of which is in the UV-A region, i.e. in the range between 320 and 400 nm. As UV-A filters, preference is given to using substances, the absorption maximum of which is between 330 and 400 nm, in particular between 350 and 380 nm. Furthermore, particular preference is given to UV-A filters which are themselves photostable or are photostabilized by further substances.

In the process according to the invention, as UV-A filters, preference is also given to using those substances which have a high specific absorption in the UV-A region, preferably a specific absorption of A 1%/1 cm (1% strength solution at 1 cm path length) at least 700, in particular of at least 900.

Examples of Oil-Soluble UV-B Filters are:

3-benzylidenecamphor derivatives, preferably 3-(4-methylbenzylidene)camphor, 4-aminobenzoic acid derivatives, preferably 2-ethylhexyl 4-(dimethylamino)benzoate, amyl 4-(dimethylamino)benzoate, polyethoxyethyl 4-bis(polyethoxy)aminobenzoate (available under the trade name Uvinul® P25 from BASF AG);
and also UV-B filters bonded to polymers (e.g. benzylidene malonate polysiloxane, INCI: Polysilicone-15).

Numerous UV-radiation-absorbing compounds are commercially available commercial products, for example the Uvinul® grades (BASF).

Uvinul® photoprotective agents comprise compounds of the classes of benzophenones, benzotriazoles, cyanoacrylates, sterically hindered amines (HALS compounds), triazines, cinnamic acid esters, para-aminobenzoates, naphthalimides. Further known UV-radiation-absorbing compounds are e.g. hydroxyphenyltriazines or oxalanilides. Such compounds are usually used alone or in mixtures with other photoprotective agents in cosmetic applications, such as, for example, in sunscreen compositions or for stabilizing organic polymers, such as, for example, plastics.

Of suitability in principle are the following UV-radiation-absorbing compounds are: substituted acrylates, such as e.g. ethyl or isooctyl α-cyano-β,β-diphenylacrylate (primarily 2-ethylhexyl α-cyano-β,β-diphenylacrylate), methyl α-methoxycarbonyl-β-phenylacrylate, methyl α-methoxycarbonyl-β-(p-methoxyphenyl)acrylate, methyl or butyl α-cyano-β-methyl-β-(p-methoxyphenyl)acrylate, N-(β-methoxycarbonyl-(β-cyanovinyl)-2-methylindoline, octyl p-methoxycinnamate, isopentyl 4-methoxycinnamate, urocanic acid or salts or esters thereof;

derivatives of p-aminobenzoic acid, in particular esters thereof, e.g. ethyl 4-aminobenzoate or ethoxylated ethyl 4-aminobenzoates, salicylates, substituted cinnamic acid esters (cinnamates), such as ethylhexyl p-methoxycinnamate or 4-isopentyl 4-methoxycinnamate, 2-phenylbenzimidazole-5-sulfonic acid or its salts.

2-Hydroxybenzophenone derivatives, such as e.g. 4-hydroxy-, 4-methoxy-, 4-octyloxy-, 4-decyloxy-, 4-dodecyloxy-, 4-benzyloxy-, 4,2′,4′-trihydroxy-, 2′-hydroxy-4,4′-dimethoxy-2-hydroxybenzophenone, and 4-methoxy-2-hydroxybenzophenonesulfonic acid sodium salt;

esters of 4,4-diphenylbutadiene-1,1-dicarboxylic acid, such as e.g. the bis(2-ethylhexyl) ester;
2-phenylbenzimidazole-4-sulfonic acid and 2-phenylbenzimidazole-5-sulfonic acid or salts thereof;
derivatives of benzoxazoles;
derivatives of benztriazoles or 2-(2′-hydroxyphenyl)benztriazoles, such as e.g. 2-(2H-benztriazol-2-yl)-4-methyl-6-(2-methyl-3-((1,1,3,3-tetramethyl-1-(trimethylsilyloxy)disiloxanyl)propyl)phenol, 2-(2′-hydroxy-5′-methylphenyl)benztriazole, 2-(3′,5′-di-tert-butyl-2′-hydroxyphenyl)benztriazole, 2-(5′-tert-butyl-2′-hydroxyphenyl)benztriazole, 2-[2′-hydroxy-5′-(1,1,3,3-tetramethylbutyl)phenyl]benztriazole, 2-(3′,5′-di-tert-butyl-2′-hydroxyphenyl)-5-chlorobenztriazole, 2-(3′-tert-butyl-2′-hydroxy-5′-methylphenyl)-5-chlorobenztriazole, 2-(3′-sec-butyl-5′-tert-butyl-2′-hydroxyphenyl)benztriazole, 2-(2′-hydroxy-4′-octyloxyphenyl)benztriazole, 2-(3′,5′-di-tert-amyl-2′-hydroxyphenyl)benztriazole, 2-[3′,5′-bis(α,α-dimethylbenzyl)-2′-hydroxyphenyl]benztriazole, 2-[3′-tert-butyl-2′-hydroxy-5′-(2-octyloxycarbonylethyl)phenyl]-5-chlorobenztriazole, 2-[3′-tert-butyl-5′-(2-(2-ethylhexyloxy)carbonylethyl)-2′-hydroxyphenyl]-5-chlorobenztriazole, 2-[3′-tert-butyl-2′-hydroxy-5′-(2-methoxycarbonylethyl)phenyl]-5-chlorobenztriazole, 2-[3′-tert-butyl-2′-hydroxy-5′-(2-methoxycarbonylethyl)phenyl]benztriazole, 2-[3′-tert-butyl-2′-hydroxy-5′-(2-octyloxycarbonylethyl)phenyl]benztriazole, 2-[3′-tert-butyl-5′-(2-(2-ethylhexyloxy)carbonylethyl)-2′-hydroxyphenyl]benztriazole, 2-(3′-dodecyl-2′-hydroxy-5′-methylphenyl)benztriazole, 2-[3′-tert-butyl-2′-hydroxy-5′-(2-isooctyloxycarbonylethyl)phenyl]benztriazole, 2,2′-methylenebis[4-(1,1,3,3-tetramethylbutyl)-6-benztriazol-2-ylphenol], the completely esterified product of 2-[3′-tert-butyl-5′-(2-methoxycarbonylethyl)-2′-hydroxyphenyl]-2H-benztriazole with polyethylene glycol 300, [R—CH2CH2-COO(CH2)3-]2 where R is 3′-tert-butyl-4-hydroxy-5′-2H-benztriazol-2-ylphenyl, 2-[2′-hydroxy-3′-(α,α-dimethylbenzyl)-5′-(1,1,3,3-tetramethylbutyl)phenyl]benztriazole, 2-[2′-hydroxy-3′-(1,1,3,3-tetramethylbutyl)-5′-(α,α-dimethylbenzyl)phenyl]benztriazole;
benzylidenecamphor or its derivatives, as are specified e.g. in DE-A 38 36 630, e.g. 3-benzylidenecamphor, 3(4′-methylbenzylidene)d-1-camphor;
α-(2-oxoborn-3-ylidene)toluene-4-sulfonic acid or its salts, N,N,N-trimethyl-4-(2-oxoborn-3-ylidenemethyl)anilinium monosulfate;
dibenzoylmethanes, such as e.g. 4-tert-butyl-4′-methoxydibenzoylmethane;
2,4,6-triaryltriazine compounds such as 2,4,6-tris{N-[4-(2-ethylhex-1-yl)oxycarbonylphenyl]amino}-1,3,5-triazine, bis(2′-ethylhexyl) 4,4′-((6-(((tert-butypaminocarbonyl)phenylamino)-1,3,5-triazine-2,4-diyl)imino)bisbenzoate;
2-(2-hydroxyphenyl)-1,3,5-triazines, such as e.g. 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-[2-hydroxy-4-(2-hydroxy-3-butyloxypropyloxy)phenyl]-4,6-bis(2,4-dimethylphenyl)-1,3,5-triazine, 2-[2-hydroxy-4-(2-hydroxy-3-octyloxypropyloxy)phenyl]-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis(2,4-dimethylphenyl)-1,3,5-triazine, 2-(2-hydroxy-4-hexyloxyphenyl)-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxypropyloxy]phenyl}-4,6-bis(2,4-dimethylphenyl)-1,3,5-triazine.

Sterically hindered amines such as, for example, N,N′-bisformyl-N,N′-bis(2,2,6,6-tetramethyl-4-piperidinyl)hexamethylenediamine (CAS No. 124172-53-8), bis(2,2,6,6-tetramethyl-4-piperidyl) sebacate (CAS No. 52829-07-9), bis(1,2,2,6,6-pentamethyl-4-piperidyl) sebacate (CAS No. 41556-26-7), methyl (1,2,2,6,6-pentamethyl-4-piperidyl)sebacate (CAS No. 82919-37-7), oligomeric sterically hindered amines which are commercially available under the trade names Uvinul® 5050 H (CAS No. 152261-33-1) and Uvinul® 5062 H (CAS No. 65447-77-0).

Further suitable UV-radiation-absorbing compounds can be found in the publication Cosmetic Legislation, vol. 1, Cosmetic Products, European Commission 1999, pp. 64-66, to which reference is hereby made.

Further suitable UV-radiation-absorbing compounds are described as well in lines 14 to 30 ([0030]) on page 6 of EP 1 191 041 A2. Reference is made to this passage in its entirety and this passage forms part of the disclosure of the present invention.

Further suitable UV-radiation-absorbing compounds are described for example on page 39, line 20 to page 41, line 10 of WO 2006/106140. Reference is made to this passage in its entirety and this passage forms part of the disclosure of the present invention.

In this connection, topical preparations are to be understood as meaning those preparations which are suitable for applying the active ingredients to the skin in a fine distribution and preferably in a form that can be absorbed through the skin. Of suitability for this are e.g. aqueous and aqueous-alcoholic solutions, sprays, foams, foam aerosols, ointments, aqueous gels, emulsions of the O/W or W/O type, microemulsions or cosmetic stick preparations.

According to one preferred embodiment of the cosmetic preparation according to the invention, the preparation comprises a carrier. A preferred carrier is water, a gas, a water-based liquid, an oil, a gel, an emulsion or microemulsion, a dispersion or mixtures thereof. The specified carriers exhibit good skin compatibility. Aqueous gels, emulsions or microemulsions are particularly advantageous for topical preparations.

Emulsifiers which can be used are nonionogenic surfactants, zwitterionic surfactants, ampholytic surfactants or anionic emulsifiers. The emulsifiers may be present in the compositions according to the invention in amounts of from 0.1 to 10% by weight, preferably 1 to 5% by weight, based on the composition.

Besides the two aforementioned groups of primary photoprotective substances, it is also possible to use secondary photoprotective agents of the antioxidant type which interrupt the photochemical reaction chain which is triggered when UV radiation penetrates into the skin. Typical examples thereof are superoxide dismutase, tocopherols (vitamin E) and ascorbic acid (vitamin C).

According to one particularly preferred embodiment, the cosmetic preparation according to the invention also comprises care substances, further cosmetic active ingredients and/or auxiliaries and additives.

The further cosmetic active ingredients used are in particular skin moisturizers, antimicrobial substances and/or deodorizing or antiperspirant substances. This has the advantage that further desired effects can be achieved which contribute to the care or treatment of the skin or, for example, increase the wellbeing of the user of the cosmetic composition when using this composition.

Thus, in the cosmetic composition, besides the carrier, the hybrid material, water and physiologically suitable solvents, inter alia, also care constituents, such as e.g. oils, waxes, fats, refatting substances, thickeners, emulsifiers and fragrances may be present. A high fraction of care substances is advantageous particularly for topical prophylactic or cosmetic treatment of the skin. It is particularly advantageous if, besides the animal and vegetable fats and oils which in many cases likewise have a care effect, the composition also comprises further care components. The group of care active ingredients which can be used comprises e.g.: fatty alcohols having 8 to 22 carbon atoms, in particular fatty alcohols of natural fatty acids; animal and vegetable protein hydrolysates, in particular elastin, collagen, keratin, milk protein, soya protein, silk protein, oat protein, pea protein, almond protein and wheat protein hydrolysates; vitamins, provitamins and vitamin precursors, in particular those of vitamin groups A and B; mono-, di- and oligosaccharides; plant extracts; honey extracts; ceramides; phospholipids; Vaseline, paraffin and silicone oils; fatty acid and fatty alcohol esters, in particular the monoesters of the fatty acids with alcohols having 3 to 24 carbon atoms.

Customary cosmetic auxiliaries which can be contemplated as additives are e.g. coemulsifiers, fats and waxes, stabilizers, thickeners, biogenic active ingredients, film formers, fragrances, dyes, pearlizing agents, preservatives, pigments, electrolytes (e.g. magnesium sulfate) and pH regulators. Suitable coemulsifiers are preferably known W/O and also O/W emulsifiers such as, for example, polyglycerol esters, sorbitan esters or partially esterified glycerides. Typical examples of fats are glycerides; waxes to be mentioned are, inter alia, beeswax, paraffin wax or microwaxes, optionally in combination with hydrophilic waxes. Stabilizers which can be used are metal salts of fatty acids such as e.g. magnesium stearate, aluminum stearate and/or zinc stearate.

Suitable thickeners are, for example, crosslinked polyacrylic acids and derivatives thereof, polysaccharides, in particular xanthan gum, guar guar, agar agar, alginates and tyloses, carboxymethylcellulose and hydroxyethylcellulose, also fatty alcohols, monoglycerides and fatty acids, polyacrylates, polyvinyl alcohol and polyvinylpyrrolidone. Biogenic active ingredients are to be understood as meaning, for example, plant extracts, protein hydrolysates and vitamin complexes. Customary film formers are, for example, hydrocolloids such as chitosan, microcrystalline chitosan or quaternized chitosan, polyvinylpyrrolidone, vinylpyrrolidone-vinyl acetate copolymers, polymers of the acrylic acid series, quaternary cellulose derivatives and similar compounds. Suitable preservatives are, for example, formaldehyde solution, p-hydroxybenzoate or sorbic acid. Suitable pearlizing agents are, for example, glycol distearic acid esters such as ethylene glycol distearate, but also fatty acids and fatty acid monoglycol esters. Dyes which can be used are the substances approved and suitable for cosmetic purposes, which are listed for example in the publication “Cosmetic Colorants” by the Dyes Commission of the German Research Society, published by Verlag Chemie, Weinheim, 1984. These dyes are usually used in concentration of 0.001 to 0.1% by weight, based on the total mixture.

An additional content of antioxidants is generally preferred. Thus, all antioxidants which are customary and suitable for cosmetic and/or dermatological applications can be used as favorable antioxidants.

The antioxidants are advantageously selected from the group consisting of amino acids (e.g. glycine, histidine, tyrosine, tryptophan) and derivatives thereof, imidazoles (e.g. urocaninic acid) and derivatives thereof, peptides such as D,L-carnosine, D-carnosine, L-carnosine and derivatives thereof (e.g. anserine), carotenoids, carotenes (e.g. β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxine, glutathione, cysteine, cystine, cystamine and the glycosyl, N-acetyl, methyl, ethyl, propyl, amyl, butyl, and lauryl, palmitoyl, oleyl, linoleyl, cholesteryl and glyceryl esters thereof) and also salts thereof, dilauryl thiodipropionate, distearyl thiodipropionate, thiodipropionic acid and derivatives (esters, ethers, peptides, lipids, nucleotides, nucleosides and salts) thereof, and also sulfoximine compounds (e.g. buthionine sulfoximines, homocysteine sulfoximines, buthionine sulfones, penta-, hexa-, heptathionine sulfoximine) in very low tolerated doses (e.g. pmol to μmol/kg), also (metal) chelating agents (e.g. hydroxy fatty acids, palmitic acid, phytic acid, lactoferrin), hydroxy acids (e.g. citric acid, lactic acid, malic acid), humic acid, bile acid, bile extracts, bilirurin, biliverdin, EDTA and derivatives thereof, unsaturated fatty acids and derivatives thereof (e.g. linolenic acid, linoleic acid, oleic acid), folic acid and derivatives thereof, ubiquinone and ubiquinol and derivatives thereof, vitamin C and derivatives thereof (e.g. ascorbyl palmitate, Mg ascorbyl phosphate, ascorbyl acetate), tocopherol and derivatives (e.g. vitamin E acetate, tocotrienol), vitamin A and derivatives (vitamin A palmitate), and coniferyl benzoate of benzoin resin, rutinic acid and derivatives thereof, glycosylrutine, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

Also advantageous are natural vegetable antioxidant complexes such as, for example, tea, grape or algae extracts, but also natural or nature-identical individual substances, such as e.g. resveratrol.

Besides the protection of the cosmetic and/or dermatological product against oxidation, antioxidants can also achieve antioxidative and also antiaging effects in the human skin.

Within the context of the invention, very particular preference is therefore given to antioxidants which penetrate into the human skin, where they efficiently develop their effect, and thereby protect, in a certain sense synergistically to the photoprotective filters, the skin against damage by UV light, against sunburn and against reactive oxygen species and free radicals. Very particular preference is given to vitamin C and vitamin E and their derivatives.

The amount of aforementioned antioxidants (one or more compounds) in the preparations is preferably 0.001 to 30% by weight, particularly preferably 0.05 to 20% by weight, in particular 1 to 10% by weight, based on the total weight of the preparation.

If vitamin E and/or derivatives thereof are used as antioxidant, it is advantageous to select their particular concentration from the range from 0.001 to 10% by weight, based on the total weight of the formulation.

If vitamin A and/or derivatives thereof or carotenoids are the antioxidant or the antioxidants, it is advantageous to select their particular concentration from the range from 0.001 to 10% by weight, based on the total weight of the formulation.

Customary oil components in cosmetics are, for example, paraffin oil, glyceryl stearate, isopropyl myristate, diisopropyl adipate, cetylstearyl 2-ethylhexanoate, hydrogenated polyisobutene, Vaseline, caprylic acid/capric acid triglycerides, microcrystalline wax, lanolin and stearic acid. However, this list is exemplary and nonexhaustive.

Natural and/or nature-identical and/or synthetic active substances with different active functions can furthermore be added to the preparations within the context of the present invention, such as, for example, caffeine for tightening the skin or promoting circulation, dihydroxyacetone and/or erythrulose for the purpose of self-tanning, bisabolol and/or panthenol for calming the skin and/or substances for enriching moisture (moisturizing), for smoothing the skin, and in particular active substances for protecting against skin aging, such as, for example, vitamin A and/or derivatives thereof, plant extracts or else protein-like substances.

Further components of cosmetic and/or dermatological preparations for the purposes of the present inventions can perform additional functions, such as e.g. the coloring of the skin in decorative cosmetics, but also that of the product itself.

Further auxiliaries and additives serve to improve the esthetic, application and/or cosmetic properties. Such auxiliaries and additives are e.g. coemulsifiers, organic solvents, superfatting agents, stabilizers, antioxidants, waxes or fats, consistency regulators, thickeners, tanning agents, vitamins, cationic polymers, biogenic active ingredients, preservatives, hydrotopes, solubilizers, dyes and fragrances.

As regards such further auxiliaries and additives, reference may be made at this point to the disclosure of EP 1455737 B, paragraphs [0075] to [0077], to which reference is hereby made in its entirety.

### EXAMPLES

Emulsifier A: emulsifier according to the invention obtained according to the process as in WO 2007/051743 from 13% by weight of polyethylene glycol PEG 6000, 57% by weight of N-vinylcaprolactam and 30% by weight of vinyl acetate with a K value of 35 (measured at 1% strength by weight in ethanol).

### Preparation of a Hydrocortisone Emulsion

### Mixture 1

### Emulsifier 10 g

Liquid paraffin 12.0 g

### Parabens 0.2 g

### Mixture 2:

Propylene glycol 8.0 g

### Hydrocortisone 1.0 g

Mixture 1 and 86.68 g of water were heated separately to 80° C. and then the water was incorporated into mixture 1 with vigorous stirring by means of a paddle stirrer at 40 rpm. Mixture 2 was heated until all of the active ingredient had dissolved, added to mixture 1 admixed with water, and the resulting mixture was then cooled to 20° C. with continuous stirring. This gives a whitish, low viscosity emulsion.

### Example 2

### Preparation of a Mint Oil Emulsion

Peppermint oil, 3.5 g

### Emulsifier A 13.8 g

### Ethanol 96% 52.0 g

### Water 30.7 g

The emulsifier was dissolved in the water at 20° C. with the help of a paddle stirrer at 1000 rpm, then admixed with the peppermint oil and then with the ethanol. This produced a clear, low viscosity emulsion.

### Example 3

### Preparation of an Oil-in-Water Emulsion

Fractionated coconut oil (Mygliol® 812) 20.0 g

### Emulsifier 20.0 g

### Water 60.0 g

According to the invention:

The emulsifier A was dissolved in water using a paddle stirrer at 1000 rpm at room temperature. The Mygliol was then stirred in and the mixture was then homogenized using an Ultra-Turrax at 20 000 rpm for 5 min.

According to the invention, this gave a low-medium viscosity white emulsion which was still stable even after 6 weeks.

For comparison, emulsions were prepared analogously using the following emulsifiers:

Kollicoat® IR (graft polymer of 75% by weight of polyvinyl alcohol and 25% by weight of PEG, MW 45 000 D): stable (6 weeks, 20° C.) white, high viscosity emulsion
Lutrol® E 400 (Macrogol 400): no emulsion formation, phase separation

Furthermore, the following emulsions were prepared for comparison in the case of an analogous composition:

cetylstearyl alcohol was melted at 60° C. and admixed with Mygliol. The water was then added at 60° C. and homogenized with the Ultra-Turrax at 20 000 rpm for 5 min. No stable emulsion was obtained; phase separation resulted during homogenization.

Tween® 80 (polyoxyethylene(20) sorbitan monooleate) was mixed with the water and heated to 40° C. Mygliol was then added and homogenized with the Ultra-Turrax at 20 000 rpm for 5 min. A white low viscosity emulsion was formed, for which, after one day at 20° C., a phase separation was observed, which was able to be reversed by redispersion.

### Example 4

### Preparation of an Emulsion of Anise Oil

Star anise oil 1.0 g

### Emulsifier A1.7 g

### Parabens 0.2 g

### Ethanol 96% 34.0 g

### Water 63.1 g

Emulsifier and parabens were dissolved in the water with stirring (paddle stirrer, 800 rpm) at 65° C. The star anise oil was then added and the mixture was further stirred for one minute. The ethanol was then incorporated with stirring.

### Example 5

Preparation of O/W Emulsions with Different Contents of Stabilizing Agents

### Formulation

Oil phase: 20 g of coconut oil (Mygliol 812)

### Emulsifier A:

### Example 5 a) 2.5 g

### Example 5b) 5.0 g

### Example 5c) 10.0 g

### Example 5d) 15 g

### Example 5e) 20 g

### Example 5f) 25 g

Water in each case ad 100 g

The emulsifier was dissolved in the respective amount of water using a paddle stirrer at 1000 rpm. The coconut oil was then stirred in and homogenized using the Ultra-Turrax for 10 min at 24 000 rpm.

The oil droplet size was determined immediately after preparation, after 3 days and after storage for 14 days at 20° C.

For comparison, emulsions were prepared analogously which comprised, as emulsifier, Kollicoat® IR, a graft copolymer of 75% by weight of polyvinyl alcohol and 25% by weight of polyethylene glycol units with an average molecular weight of 45 000 daltons.

The particle sizes of the oil droplets was determined using a Malvern Mastersizer 2000.

The table below gives the average particle sizes d(0.5) in μm determined directly after preparation and also after storage for 3 and 14 days at 20° C. The left-hand column in each case gives the fraction of emulsifier based on the total weight of the emulsion in

| | Emulsifier A, monomodal | | | Kollicoat IR, bimodal | | |
|---|---|---|---|---|---|---|
| | After | | | After | | |
| | preparation | 3 days | 14 days | preparation | 3 days | 14 days |
| 2.5 | 6.164 | — | 6.873 | 3.471 | — | 83.750 |
| 5 | 4.561 | — | 4.360 | 2.731 | — | 4.986 |
| 10 | 2.818 | — | 2.954 | 1.073 | — | 2.055 |
| 15 | 2.076 | 2.070 | 2.085 | 3.023 | 12.274 | 56.053 |
| 20 | 1.376 | 1.238 | 1.330 | 0.873 | 16.321 | 21.348 |
| 25 | 2.556 | 2.467 | 2.495 | 1.172 | 1.241 | 5.335 |

The emulsions according to the invention are monomodal and very stable as regards their oil droplet size even upon storage. The comparison emulsions all have a bimodal distribution after preparation. The size and distribution of the oil droplets vary considerably at the different storage times.

The particle size distribution of formulation 5d) is shown graphically in the figure.

1. A method of making emulsions comprising polymerizing vinyl acetate and N vinyllactams in the presence of a polyether, and using the copolymers as stabilizing agents for emulsions.

2. The method according to claim 1, where the copolymers are obtained by radically initiated polymerization of a mixture of
i) 30 to 80% by weight of N-vinyllactam,
ii) 10 to 50% by weight of vinyl acetate and
iii) 10 to 50% by weight of a polyether,
with the proviso that the sum of i), ii) and iii) is equal to 100% by weight.

3. The method according to claim 1, where the copolymers are obtained from:
i) 30 to 70% by weight of N-vinyllactam
ii) 15 to 35% by weight of vinyl acetate, and
iii) 10 to 35% by weight of a polyether.

4. The method according to claim 1, where the copolymers are obtained from:
i) 40 to 60% by weight of N-vinyllactam
ii) 15 to 35% by weight of vinyl acetate
iii) 10 to 30% by weight of a polyether.

5. The method according to claim 1, wherein the N-vinyllactam comprises N vinylcaprolactam or N vinylpyrrolidone or mixtures thereof.

6. The method according to claim 5, where the N-vinyllactam comprises N vinylcaprolactam.

7. The method according to claim 1, where the polyether comprises a polyalkylene glycol having a molecular weight in the range of from 1000 to 100 000 daltons.

8. The method according to 1, where the water-soluble or water-dispersible copolymers have K values in the range from 10 to 60, measured in a 1% strength by weight ethanolic solution.

9. The method according se according to claim 8, where the water-soluble or water-dispersible copolymers have K values in the range from 15 to 40, measured in a 1% strength by weight ethanolic solution.

10. The method according to claim 1, where the emulsions are of the oil-in-water type.

11. The method according to claim 1, where the emulsions have average droplet sizes d(0.5) of from 0.1 to 50 μm.

12. The method according to claim 11, where the emulsions have average droplet sizes d(0.5) of from 0.5 to 25 μm.

13. The method according to claim 1, where the oil phase comprises oil-soluble active ingredients.

14. An emulsion comprising, as stabilizing agents, water-soluble or water-dispersible copolymers which are obtained by polymerization of vinyl acetate and N vinyllactams in the presence of a polyether.

15. A pharmaceutical or cosmetic preparation obtained comprising an emulsion according to the method of claim 1.

16. A pharmaceutical or cosmetic preparation comprising the emulsion of claim 14.
